(19) United States
(12) Patent Application Publication
(10) Pub. No.: US 2026/0275260 A1
(43) Pub. Date: Sep. 17, 2026

(54) Flexible Facility Configurations for Therapeutic Product Manufacturing

(71) Applicant: AMGEN INC., Thousand Oaks, CA (US)

(72) Inventors: Jeffrey T. Ranney, Thousand Oaks, CA (US); James Thomas Weidner, Westlake Village, CA (US); Robert James Gorski, Kirkland, VA (US)

(21) Appl. No.: 18/877,265

(22) PCT Filed: Jun. 27, 2023
(86) PCT No.: PCT/US2023/026277

§ 371 Date: Dec. 19, 2024

Related U.S. Application Data
(60) Provisional application No. 63/356,361, filed on Jun. 28, 2022.

(51) Int. Cl. C12M 1/00

(52) U.S. Cl. CPC C12M 23/28; C12M 23/26; C12M 23/34; C12M 47/12

(57) ABSTRACT

Disclosed herein are flexible facilities for manufacturing at least one therapeutic product, where the flexible facilities include a reconfigurable, single-use area and a stainless steel area that may be optionally segregated by a partition.

### CROSS-REFERENCE TO RELATED APPLICATION

Priority is claimed to U.S. Provisional Patent Application No. 63/356,361, filed Jun. 28, 2022, the entire contents of which are incorporated herein by reference.

### FIELD OF DISCLOSURE

The present disclosure relates to flexible manufacturing facilities that enable the production of therapeutic products, such as, e.g., drug substances, cell therapies, and the like, that may require different equipment, such as, e.g., single use systems and stainless steel equipment, in sequential or concurrent campaigns. In some embodiments, the flexible manufacturing facility combines a reconfigurable, single-use area with a traditional stainless steel area, wherein the reconfigurable, single-use area and the stainless steel area are optionally segregated by a partition.

### BACKGROUND

Biologics are used worldwide in a variety of therapeutic areas, ranging from oncology to cardiology and beyond. However, the costs associated with manufacturing biologics are high due to their complex production methods, as well as the single-modality and/or single-product focus of a typical biomanufacturing facility. Biologics manufacturing typically utilizes multistep processes that include cell culture scale up, large scale production cell culture, and purification of the desired biologic following cell harvest; these steps are often unique to a given modality or product. Manufacturing complexity has increased in recent years due to the availability of numerous novel modalities such as monoclonal antibodies, BiTE® molecules, fusion proteins, CAR-T cell therapies, RNA therapeutics, and the like. While improvements in all facets of production have reduced commercial-scale manufacturing expenses for many biologics, the upfront costs associated with building and maintaining a biomanufacturing facility, including the upfront costs associated with campaign changes to alter or expand the types of modalities and/or products manufactured in a given facility, can still be prohibitive. Accordingly, there is a need in the art for novel, reconfigurable biomanufacturing facility configurations that enable multiple products and/or modalities to be manufactured in a single facility throughout its lifetime at reduced cost and in reduced time to facilitate long range supply planning.

Biologics are commonly produced in an industry standard platform referred to as “Fed Batch.” Fed-batch processes employ partly open systems in which one or more nutrient boluses are fed to a bioreactor during cell culture. Fed-batch production processes are often performed in large stainless steel bioreactors, and much of the expense associated with biopharmaceutical manufacturing can be attributed to the capital investments required to build and maintain the infrastructure needed to support the production and purification of a particular biologic. Illustratively, building a Fed Batch facility often requires approximately $1.5 billion in capital investment and approximately 6 years for licensure. In addition, traditional structures for manufacturing biologic products, such as monoclonal antibodies and microbial products, do not allow for efficient use of facility space, e.g., due to the need for large stainless steel tanks and extensive stainless steel hard-piping to support fed-batch processes. Furthermore, the layout of traditional biomanufacturing facilities, which often includes extensive hard-piping and sizable equipment, can limit their ability to be readily re-configured or expanded to manufacture a new product line. This limitation is compounded by the diverse platforms used to manufacture biologics, which are often product specific and lack interchangeability.

An ideal biomanufacturing facility would be inexpensive to build and capable of rapid expansion and reconfiguration to integrate new products and processes quickly and cost effectively. Moreover, it would be advantageous for such a manufacturing facility to be able to maintain and improve upon the high level of quality required for current good manufacturing practice (cGMP) for drug manufacture (e.g., conformance with 21 C.F.R. Part 11).

Single-use production technologies, which simplify equipment and facility design relative to reusable systems that require cleaning and sterilization between each production batch, are increasingly popular in biomanufacturing facilities. Single-use components do not require cleaning after every batch and are a cost-effective means for manufacturing biologics. However, the manufacturing requirements of certain biologics exceed the current volumetric or other capacities achievable with single-use technologies. Accordingly, for certain biologics with high quantity requirements, reusable, high volume stainless-steel vessels cannot be avoided. Such vessels must be connected by stainless steel piping to other unit operations, media and buffer supply, water supply, and clean-in-place and steam-in-place systems. As noted above, the fabrication and installation of these stainless-steel vessels, and all the utilities that support them, is expensive and requires considerable lead time for design and manufacture.

Thus, there is a need for a flexible manufacturing facility that allows a user to efficiently deploy common resources between product lines and that can be readily modified to accommodate a new manufacturing process or a new manufacturing line with reduced facility downtime.

### SUMMARY

The present disclosure provides a flexible manufacturing facility for the production of biologics, wherein the flexible manufacturing facility can be tailored to the specific needs of one or more biologics, such as, e.g., one or more biologics of different modalities. This novel facility arrangement utilizes a reconfigurable, single use area and a stainless steel area that may be optionally segregated by a partition.

Disclosed herein is a flexible facility for manufacturing at least one therapeutic product, comprising:
a reconfigurable, single-use area comprising at least one single-use bioreactor, wherein one or more of the at least one single use bioreactor(s) has a volume of at least 1000 L (e.g., at least 2000 L, greater than 2000 L); and
a stainless steel area comprising at least one stainless steel bioreactor, wherein each stainless steel bioreactor in the stainless steel area has a volume greater than 5000 L,
wherein one or more of the at least one stainless steel bioreactor(s) is fluidly connected to one or more of the at least one single use bioreactor(s).

In some embodiments, one or more of the at least one single use bioreactor(s) has a volume of 1000 L. In some embodiments, a plurality of the at least one single use bioreactor(s) has a volume of 1000 L.

In some embodiments, one or more of the at least one single use bioreactor(s) has a volume of at least 2000 L. In some embodiments, one or more of the at least one single use bioreactor(s) has a volume of 2000 L. In some embodiments, a plurality of the at least one single use bioreactor(s) has a volume of 2000 L.

In some embodiments, one or more of the at least one single use bioreactor(s) has a volume of at least 3000 L. In some embodiments, one or more of the at least one single use bioreactor(s) has a volume of 3000 L. In some embodiments, a plurality of the at least one single use bioreactor(s) has a volume of 3000 L.

In some embodiments, one or more of the at least one single use bioreactor(s) has a volume of at least 5000 L. In some embodiments, one or more of the at least one single use bioreactor(s) has a volume of 5000 L. In some embodiments, a plurality of the at least one single use bioreactor(s) has a volume of 5000 L.

In some embodiments, one or more of the at least one single use bioreactor(s) has a volume of 1000 L, 2000 L, 3000 L, or 5000 L. In some embodiments, a plurality of the at least one single use bioreactor(s) have volumes independently chosen from 1000 L, 2000 L, 3000 L, and 5000 L.

In some embodiments, one or more of the at least one single use bioreactor(s) has a volume of 200 L. In some embodiments, one or more of the at least one single use bioreactor(s) has a volume of 500 L.

Non-limiting examples of equipment that can be employed in the flexible facility include bioreactors, disc stack centrifuges, single use centrifuges, tangential flow filtration (TFF) skids, depth filtration skids, in-line dilution skids, chromatography columns with associated control equipment, media tanks, harvest tanks, purification vessels, depth filter holders, water softening and/or de-chlorination systems, clean steam generators, water for injection (WFI) storage tanks, WFI break tanks, WFI stills, cooling towers, switchboards, emergency generators, chillers, pumps, autoclaves, air handling units, process waste neutralization (such as, e.g., fiberglass reinforced plastic (FRP)) equipment, biowaste collection and/or inactivation systems, clean-in-place systems, steam-in-place systems, parts washers, and/or other equipment.

By way of non-limiting examples and without limitation, U.S. Publication Nos. 2013/0280797, 2012/0077429, 2011/0280797, and 2009/0305626 and U.S. Pat. Nos. 8,298,054; 7,629,167; and 5,656,491, which are hereby incorporated by reference, describe example facility areas, equipment, and/or systems having features that may be suitable for use in the flexible facilities described herein.

In some embodiments, the stainless steel area, taken as a whole, has a lower air classification than the reconfigurable, single-use area, taken as a whole. In some embodiments, the stainless steel area, taken as a whole, has lower architectural finishes than the reconfigurable single-use area, taken as a whole. In some embodiments, the stainless steel area, taken as a whole, has a lower air classification and lower architectural finishes than the reconfigurable, single-use area, taken as a whole.

In some embodiments, the stainless steel area is an unclassified manufacturing space. In some embodiments, at least a portion of the reconfigurable, single-use area (e.g., a seed train area; a purification area) is a classified manufacturing space. The integrity and environmental control inside at least a portion of the reconfigurable, single-use area facilitates the use of an unclassified manufacturing space.

In some embodiments, one or more of the at least one stainless steel bioreactor(s) is supported on a ground surface.

In some embodiments, one or more of the at least one stainless steel bioreactor(s) may be suspended from the structure itself.

In some embodiments, the stainless steel area comprises a plurality of stainless-steel bioreactors each having a volume of at least 10,000 L.

In some embodiments, each stainless steel bioreactor in the stainless steel area has a volume between 5000 L and 20,000 L. In some embodiments, each stainless steel bioreactor in the stainless steel area has a volume between 5000 L and 15,000 L. In some embodiments, each stainless steel bioreactor in the stainless steel area has a volume between 5000 L and 10,000 L.

In some embodiments, the stainless steel area has no physical barrier separating it from the reconfigurable, single-use area.

In some embodiments, the reconfigurable, single-use area and the stainless steel area are segregated by a partition. In some embodiments, the partition is a wall. In some embodiments, the partition is a plastic sheet.

In some embodiments, the stainless steel area is completely enclosed. In some embodiments, the stainless steel area is completely enclosed by one or more walls and a ceiling.

In some embodiments, the stainless steel area has separate air handling from the reconfigurable, single-use area.

In some embodiments, a portion of the at least one stainless steel bioreactor(s) penetrates the wall and is fluidly connected through tubing to one or more of the at least one single use bioreactor(s). In some embodiments, the portion is sufficient to facilitate connections between instrumentation and/or process units. In some embodiments, the portion is minimally sufficient to facilitate connections between instrumentation and/or process units.

In some embodiments, at least 1% (e.g., at least 2%, at least 3%, at least 4%, at least 5%; 1%, 2%, 3%, 4%, 5%) of the exterior vessel surface area of the at least one stainless steel bioreactor(s) penetrates the wall and is fluidly connected through tubing to one or more of the at least one single use bioreactor(s). In some embodiments, 3% to 7% (e.g., 3.5% to 6.5%, 4% to 6%, 4.5% to 5.5%; 3%, 4%, 5%, 6%, 7%) of the exterior vessel surface area of the at least one stainless steel bioreactor(s) penetrates the wall and is fluidly connected through tubing to one or more of the at least one single use bioreactor(s).

In some embodiments, the portion penetrating the wall comprises one or more components chosen from probe belts to allow access to bioreactor instrumentation, connections for perfusion, and connections for feed solutions (e.g., nutrient feeds; anti-foam containing solutions).

In some embodiments, the fluid connection is configured to enable bypass of the stainless steel area. In some embodiments, the fluid connection is removable. In some embodiments, the fluid connection is capable of being moved between different bioreactors within the flexible facility.

In some embodiments, the stainless steel area further comprises a harvest area. In some embodiments, the harvest area in the stainless steel area includes one or more centrifuges. In some embodiments, the harvest area in the stainless steel area includes one or more stainless steel centrifuges. In some embodiments, the equipment in the stainless steel area is connected with tubing and/or one or more valves to allow the fluid connections needed to complete the specific process being run. In some embodiments, one or more valves are used to bypass certain equipment that is not needed for the process being run. In some embodiments, one or more valves are used to shut off fluid flow to allow changing of equipment. For example, in some embodiments, into each stainless steel bioreactor, there is connected an inlet conduit from the seed train. In some embodiments, from each stainless steel bioreactor, there is connected an outlet conduit to a centrifuge for harvesting.

In some embodiments, the reconfigurable, single-use area may comprise one or more additional spaces independently chosen from cell culture units, pre-viral units, post-viral units, utility yards, warehouses, media buffer facilities, offices, personnel units, and production units. A production unit may be useful for manufacturing a therapeutic product.

In some embodiments, the reconfigurable, single-use area may comprise a reagent preparation area, a seed train area, and a purification area.

In some embodiments, the reconfigurable, single-use area further comprises equipment for performing downstream purification. In some embodiments, the equipment for performing downstream purification is single use or stainless steel. In some embodiments, the equipment for performing downstream purification is single use. In some embodiments, the equipment for performing downstream purification is stainless steel. In some embodiments, some of the equipment for performing downstream purification is stainless steel and some of the equipment for performing downstream purification is single use.

In some embodiments, the stainless steel area further comprises equipment for performing downstream purification. In some embodiments, the equipment for performing downstream purification is single use or stainless steel. In some embodiments, the equipment for performing downstream purification is single use. In some embodiments, the equipment for performing downstream purification is stainless steel.

In some embodiments, the reconfigurable, single-use area further comprises one or more centrifuges. In some embodiments, the one or more centrifuges are single use and/or stainless steel equipment. In some embodiments, the one or more centrifuges are single use equipment. In some embodiments, the one or more centrifuges are stainless steel equipment.

In some embodiments, the stainless steel area further comprises one or more centrifuges. In some embodiments, the one or more centrifuges are single use and/or stainless steel equipment. In some embodiments, the one or more centrifuges are single use equipment. In some embodiments, the one or more centrifuges are stainless steel equipment.

In some embodiments, the reconfigurable, single-use area comprises a reagent preparation area. In some embodiments, the reconfigurable, single-use area includes walls around the reagent preparation area. In some embodiments, the reagent preparation area has separate air handling. In some embodiments, the reconfigurable, single-use area comprises a seed train area. In some embodiments, the reconfigurable, single-use area includes walls around the seed train area. In some embodiments, the seed train area has separate air handling. In some embodiments, the reconfigurable, single-use area comprises a purification area. In some embodiments, the reconfigurable, single-use area includes walls around the purification area. In some embodiments, the purification area has separate air handling.

In some embodiments, the reconfigurable, single-use area comprises a reagent preparation area, a seed train area, and a purification area. In some embodiments, the reconfigurable, single-use area comprises a reagent preparation area, a seed train area, and a purification area, along with one or more partitions at least partially segregating one or more of the reagent preparation area, the seed train area, and the purification area. In some embodiments, the reconfigurable, single-use area comprises a reagent preparation area, a seed train area, and a purification area, wherein the reagent preparation area, the seed train area, and the purification area are not all serviced by the same air handling system.

In some embodiments, the stainless steel area is adjacent to the seed train area or adjacent to the purification area or adjacent to both the seed train area and the purification area.

In some embodiments, the reconfigurable, single-use area comprises one or more seed trains. In some embodiments, the reconfigurable, single-use area comprises one or more wave bioreactors, e.g., one or more wave bioreactors having a volume of at least 50 L (e.g., 50 L). In some embodiments, the reconfigurable, single-use area comprises one or more seed bioreactors, such as, e.g. one or more seed bioreactors with a volume of at least 500 L (e.g., 500 L) or a volume of at least 2,000 L (e.g., 2,000 L).

In some embodiments, the reconfigurable, single-use area further comprises one or more purification skids. In some embodiments, each of the one or more purification skids comprises one or more components independently chosen from chromatography skids, chromatography columns, viral inactivation systems, viral filtration systems, ultrafiltration diafiltration systems, and bulk filtration systems. In some embodiments, the reconfigurable, single-use area further comprises a chromatography skid. In some embodiments, the reconfigurable, single-use area further comprises a viral inactivation system. In some embodiments, the reconfigurable, single-use area further comprises a viral filtration system. In some embodiments, the reconfigurable, single-use area further comprises an ultrafiltration/diafiltration system. In some embodiments, the reconfigurable, single-use area further comprises a bulk filtration system. In some embodiments, the reconfigurable, single-use area further comprises a viral inactivation system and a viral filtration system.

In some embodiments, one or more purification skids in the flexible facility have separate air handling.

In some embodiments, the flexible facility comprises a plurality of connections capable of being reconfigured to connect different equipment (e.g., bioreactors, purification skids, etc.) in the flexible facility.

In some embodiments, utilities are provided overhead, from one or more wall(s), or from one or more floor mounted utility distribution(s), or a combination of the aforementioned utility distribution options in the reconfigurable, single-use area. In some embodiments, utilities are provided overhead in the reconfigurable, single-use area. In some embodiments, utilities in the reconfigurable, single-use area are readily accessible.

In some embodiments, a plurality of equipment in the reconfigurable, single-use area is capable of being moved around the reconfigurable, single-use area.

In some embodiments, the flexible facility further comprises quality assurance (QA) sections, administrative/office sections, plant utilities (e.g., air-handling, electrical, and plumbing), quality control areas, laboratory areas, and receiving areas.

In some embodiments, the flexible facility further comprises one or more additional spaces independently chosen from utility spaces, media buffer facilities, personnel changing rooms, and production units. In some embodiments, the flexible facility further comprises a utility space. In some embodiments, the flexible facility further comprises a media buffer facility. In some embodiments, the flexible facility further comprises a personnel changing room. In some embodiments, the flexible facility further comprises a production unit. In some embodiments, the flexible facility further comprises a utility space, a media buffer facility, a personnel changing room, and a production unit. In some embodiments, the at least one additional space (e.g., a utility space, a media buffer facility, a personnel changing room, and/or a production unit) is located within the reconfigurable, single-use area.

In some embodiments, the flexible facility comprises at least one of a fermentation unit, a pre-viral unit, a post-viral unit, a utility space, a warehouse, a media buffer facility, an office, a personnel unit, and a production unit.

In some embodiments, any typical manufacturing and clean room equipment can be included in the flexible facility.

In some embodiments, the flexible facility is configured for producing one or more therapeutic products independently chosen from monoclonal antibodies, BiTE@ molecules, RNA-based drug substances, cell therapy products, and fermentation products. In some embodiments, the flexible facility is configured for producing a monoclonal antibody. In some embodiments, the flexible facility is configured for producing a BiTE@ molecule. In some embodiments, the flexible facility is configured for producing an RNA-based drug substance. In some embodiments, the flexible facility is configured for producing a cell therapy product. In some embodiments, the flexible facility is configured for producing a fermentation product.

In some embodiments, the stainless steel bioreactor(s) in the stainless steel area are devoted to a single therapeutic product. In some embodiments, the entire flexible facility is devoted to production of a single therapeutic product. In some embodiments, the stainless steel bioreactor(s) in the stainless steel area contain only one therapeutic product-producing cell line.

In some embodiments, the flexible facility is devoted to production of more than one (e.g., two, three, four, five, six, seven, eight, nine, ten, eleven, twelve, etc.) therapeutic product at the same time (e.g., with multiple products in, e.g., a seed train, production bioreactors, and purification skids, at the same time). In some embodiments, the flexible facility is devoted to production of two therapeutic products at the same time. In some embodiments, the flexible facility is devoted to production of three therapeutic products at the same time. In some embodiments, the flexible facility is devoted to production of four therapeutic products at the same time. In some embodiments, the flexible facility is devoted to production of five therapeutic products at the same time. In some embodiments, the flexible facility is devoted to production of six therapeutic products at the same time. In some embodiments, the flexible facility is devoted to production of seven therapeutic products at the same time. In some embodiments, the flexible facility is devoted to production of eight therapeutic products at the same time. In some embodiments, the flexible facility is devoted to production of nine therapeutic products at the same time. In some embodiments, the flexible facility is devoted to production of ten therapeutic products at the same time. In some embodiments, the flexible facility is devoted to production of eleven therapeutic products at the same time. In some embodiments, the flexible facility is devoted to production of twelve therapeutic products at the same time.

In some embodiments, during a campaign change, more than one (e.g., two, three, four, five, six, seven, eight, nine, ten, eleven, twelve, etc.) therapeutic product is produced in the flexible facility. For example, in some embodiments, after cell harvest and during purification, production of a second therapeutic product could be initiated in a seed train. In some embodiments, the stainless steel bioreactors in the stainless steel area contain more than one therapeutic product-producing cell line.

In some embodiments, the flexible facility is devoted to production of more than one (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43,44, 45, 46, 47, 48, 49, 50, etc.) therapeutic product during its lifetime. In some embodiments, the flexible facility is devoted to production of up to 10 therapeutic products during its lifetime. In some embodiments, the flexible facility is devoted to production of up to 15 therapeutic products during its lifetime. In some embodiments, the flexible facility is devoted to production of up to 20 therapeutic products during its lifetime. In some embodiments, the flexible facility is devoted to production of up to 25 therapeutic products during its lifetime. In some embodiments, the flexible facility is devoted to production of up to 30 therapeutic products during its lifetime. In some embodiments, the flexible facility is devoted to production of up to 35 therapeutic products during its lifetime. In some embodiments, the flexible facility is devoted to production of up to 40 therapeutic products during its lifetime. In some embodiments, the flexible facility is devoted to production of up to 45 therapeutic products during its lifetime. In some embodiments, the flexible facility is devoted to production of up to 50 therapeutic products during its lifetime.

In some embodiments, the flexible facility is configured to support cGMP (current good manufacturing practice) processes. In some embodiments, the flexible facility is configured to support GMP processes. In some embodiments, the flexible facility is configured to support non-GMP processes. In some embodiments, the flexible facility supports the production of a GMP clinical product. In some embodiments, the flexible facility supports the production of a GMP commercial product. In some embodiments, the flexible facility supports the production of a non-GMP product. In some embodiments, the flexible facility supports the production of a GMP clinical product and a GMP commercial product. In some embodiments, the flexible facility supports the production of a GMP clinical product and a non-GMP product. In some embodiments, the flexible facility supports the production of a GMP commercial product and a non-GMP product. In some embodiments, the flexible facility supports the production of a GMP clinical product, a GMP commercial product, and a non-GMP product.

### BRIEF DESCRIPTION OF THE DRAWINGS

FIG. 1 depicts a non-limiting example of a flexible manufacturing facility according to the present disclosure. FIG. 1 is a top plane view of an example embodiment of a flexible facility. In some embodiments, the flexible facility of FIG. 1 can be constructed in a series of phases, such as a first phase, a second phase, and subsequent phases.

FIG. 2A is a schematic diagram depicting various components of the flexible manufacturing facility of FIG. 1.

FIG. 2B is a schematic diagram depicting components of the portion of the at least one stainless steel bioreactor of FIGS. 1 and 2A.

FIG. 2C is a schematic diagram depicting at least one purification skid of the one or more purification skids of FIGS. 1 and 2A.

### DETAILED DESCRIPTION

The present disclosure relates to a new manufacturing facility configuration which integrates industry-leading technology from a next-generation biomanufacturing platform based on single-use equipment to enable intensified processes within a traditional Fed Batch facility based on reusable stainless steel equipment. The new facility configuration may facilitate the production of multiple biologics, concurrently or sequentially, manufactured using diverse manufacturing platforms. This facility configuration also provides enhanced flexibility relative to traditional biomanufacturing facility layouts. For example, the facility can be configured or re-configured to support any of the following manufacturing technologies: (a) Fed Batch cell culture processing (e.g., up to 10,000 L scale, at greater than 10,000 L scale) (e.g., utilizing single-use and/or stainless steel equipment); (b) single-use cell culture processing up to 2,000 L scale (e.g., utilizing only single-use technologies); and (c) continuous upstream manufacturing up to 500 L scale.

In addition to providing platform flexibility, biomanufacturing facilities described herein are expected to exhibit one or more of the following advantages when compared to a traditional Fed Batch facility with similar production capacity: (a) reduced financial risk (e.g., with less than 50% capital outlay) due to lower space requirements, lower capacity equipment, less stainless steel hard-piping, and fewer automated stainless steel valves; (b) faster construction with earlier licensure due to improved space utilization; and (c) reduced carbon emissions and water consumption (e.g., by up to 50%) when sustainable building materials are employed and reliance on stainless steel equipment requiring regular cleaning and sterilization procedures is reduced.

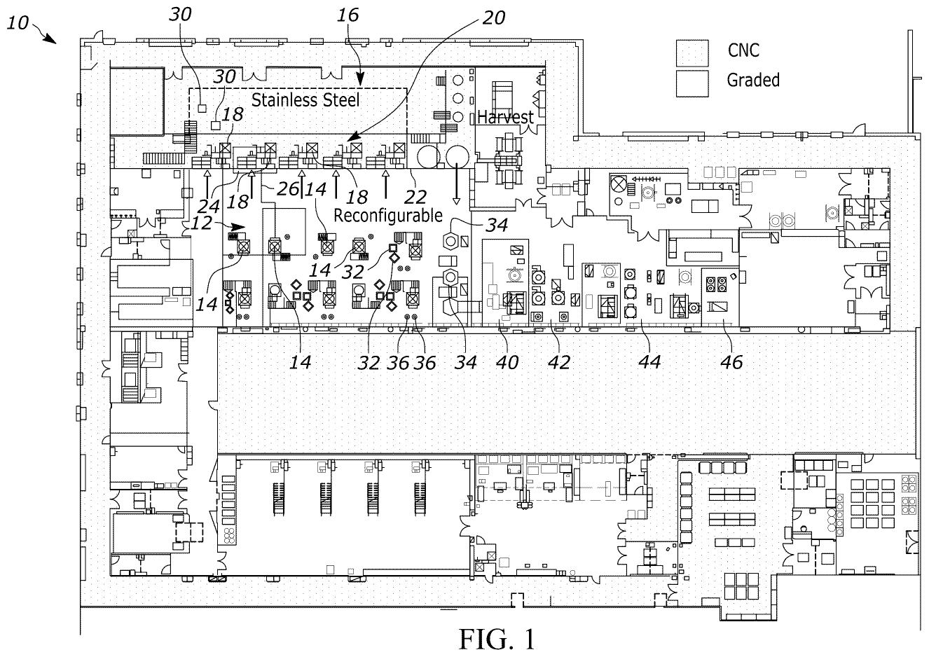

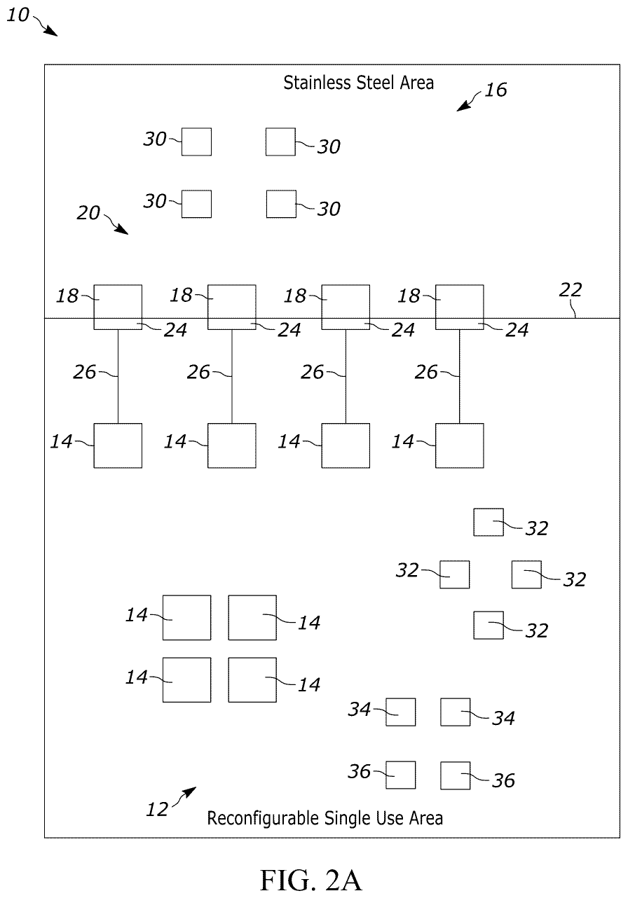

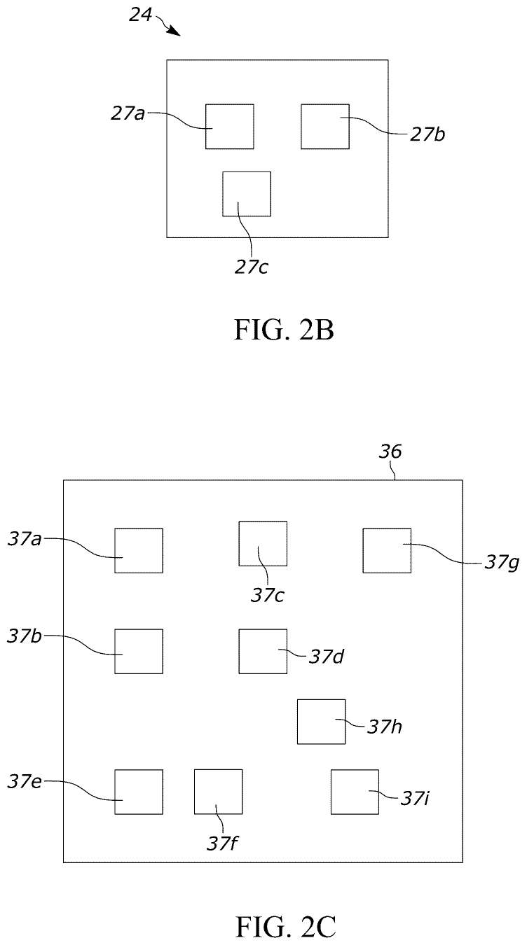

Referring now to FIGS. 1-2C, a flexible manufacturing facility 10 for manufacturing at least one therapeutic product is depicted. The flexible facility 10 includes a reconfigurable, single-use area 12 comprising at least one single-use bioreactor 14. In this embodiment, and as explained more below, one or more of the at least one single-use bioreactor(s) 14 has a volume of at least 1000 L. In another example, the one or more of the at least one single-use bioreactor(s) 14 has a volume of at least 2000 L and still falls within the scope of the present disclosure.

The flexible facility 10 also includes a stainless steel area 16 comprising at least one stainless steel bioreactor 18. Each stainless steel bioreactor 18 in the stainless steel area 16 has a volume greater than 5000 L. In addition, and as also explained more below, one or more of the at least one stainless steel bioreactor(s) 18 can be fluidly connected to one or more of the at least one single-use bioreactor(s) 14. In another example, and as depicted in FIG. 1, the stainless steel area 16 includes a plurality of stainless steel bioreactors 20, each having a volume of at least 10,000 L.

In one example, the at least one stainless steel bioreactor 18 is a large scale stainless steel bioreactor and includes at least one valve assembly. The at least one stainless steel bioreactor 18 may include a steam-in-place bioreactor with large scale capacity, e.g., configured to hold a volume of fluid of up to 10,000 L or more in one example and as noted. The at least one valve assembly of the steam-in-place large scale bioreactor enables the steam-in-place large scale bioreactor to be completely sterilized before cell culture (e.g., perfusion cell culture) begins. Specifically, the at least one valve assembly may include a plurality of valves and steam, such as clean steam CS, flows into a one valve, upwardly into other valves and to a port.

Additionally, after flowing into the first valve, the steam also flows downwardly into yet another valve and through a steam trap to sterilize the bioreactor and create a steam sterilized aseptic environment. In addition, process waste PW flows out of some of the valves during cleaning, emptying into a drain, as is understood by those having ordinary skill in the art. After use, the steam-in-place bioreactor is cleaned in-place and the steam-in-place process of sterilization occurs again before any further use. The at least one valve assembly also includes the port for coupling to an aseptic connector valve assembly, such as an autoclaved aseptic connector valve assembly, or other device. The aseptic connector valve assembly enables sterilized connectivity of a single use perfusion device or a reusable perfusion device to the at least one stainless steel bioreactor 18, such as the steam-in-place large scale bioreactor. In addition, the at least one valve assembly enables sterilized connectivity of the single-use bioreactor 14 to the at least one stainless steel bioreactor 18.

In another example, the at least one stainless steel bioreactor 18 may include a side and a first valve assembly, such as an autoclaved valve assembly, coupled to the side of the at least one stainless steel bioreactor 18. The autoclaved valve assembly may include a plurality of valves and steam, such as clean steam CS, flows into a first valve, upwardly to other valves and to a port. Additionally, after flowing into the first valve, steam flows downwardly through yet another valve and through a steam trap to sterilize the at least one stainless steel bioreactor 18 and create a steam-in-place process of sterilization before further use, for example. In this example, there may be only at least one aseptic connector coupled to a valve assembly, such as an autoclaved valve assembly. This autoclaved valve assembly enables connection via the at least one aseptic connector of the autoclaved valve assembly to a factory assembled and irradiated perfusion device or an autoclave sterilized perfusion device, which may be a single use perfusion device or a reusable perfusion device, for example. In addition, multiple perfusion devices are able to be aseptically coupled to the at least one stainless steel bioreactor 18 without additional steam sterilization. Further, the autoclaved valve assembly also enables connectivity of the at least one single-use bioreactor 14 to the at least one stainless steel bioreactor 18. In yet another example, the at least one stainless steel bioreactor 18 may enable connection of an autoclaved perfusion device and cleaning-in-place of a valve assembly, but without the use of an aseptic connector. Instead, a hose assembly may be used an as alternate connector to couple the at least one stainless steel bioreactor 18 to an autoclaved perfusion device and/or the at least one single-use bioreactor 14.

As further depicted in FIGS. 1 and 2A, in another example, the reconfigurable, single-use area 12 and the stainless steel area 16 are segregated by a partition 22. In one example, the partition is a wall. In another example, the stainless steel area 16 is completely enclosed with separate air handling, as explained more below. In addition, a portion 24 of the at least one stainless steel bioreactor(s) 18 penetrates the partition wall 22 and is fluidly connected through tubing 26 to one or more of the at least one single-use bioreactor(s) 14. The fluid connection is configured to enable bypass of the stainless steel area 16, as also explained more below.

In another example, the at least one stainless steel bioreactor 18 includes an adapter assembly, such as a single use adapter assembly and/or a wye assembly, that is configured to be and/or is coupled to an aseptic connector. The adapter assembly, such as the wye assembly, includes a connector that is directly coupled to the aseptic connector, which is ultimately coupled to a pair of tubes, such as the tubing 26. Each of the aseptic connector or connectors of the wye assembly are configured to be coupled to a reusable or single use perfusion device or the at least one single-use bioreactor 14 of the reconfigurable single-use area 12 of FIGS. 1 and 2A, for example. This enables multiple perfusion devices or multiple single-use bioreactors 14 to be operatively coupled to the at least one stainless steel bioreactor 18, for example.

In another example, the portion 24 of the at least one stainless steel bioreactor 18 penetrating the partition wall 22 comprises one or more components, including but not limited to, probe belts 27a, perfusion connections for perfusion 27b, and feed solution tank connections 27c, as depicted in FIG. 2B.

As depicted in FIGS. 1 and 2A, and in one example, the stainless steel area 16 includes one or more centrifuges 30. In addition, the reconfigurable, single-use area 12 also includes one or more centrifuges 32. In another example, the reconfigurable, single-use area 12 includes one or more seed trains 34. In yet another example, the reconfigurable, single-use area 12 includes one or more purification skids 36. In one example, and as depicted in FIG. 2C, each of the one or more purification skids 36 comprises one or more components independently chosen from and/or including chromatography skids 37a, chromatography columns 37b, viral inactivation systems 37c, viral filtration systems 37d, ultrafiltration diafiltration systems 37e, bulk filtration systems 37f, conjugation tank systems 37g, oxidation tank systems 37h, and reduction tank systems 37i.

In some embodiments, the flexible facility 10 of the present disclosure exhibits or is configured for one or more of the following properties:
1) Extensive deployment of reconfigurable space to accommodate cell culture processing (e.g., high titer (such as, e.g., 1 to 15 g/L titers or more) at the 10,000 L scale with even higher titers (such as, e.g., up to 30 g/L or more) at the 2,000 L scale. Fed-batch cultures and perfusion cultures can be accommodated in one facility, including as part of a single therapeutic product manufacturing process.
2) Inclusion of stainless steel bioreactors 18 (such as, e.g., a 10,000 L scale stainless steel production bioreactor) that maximize purification capacity using high binding capacity, commercially available chromatography resins.
3) Inclusion of stainless steel bioreactors 18 (such as, e.g., a 10,000 L scale stainless steel production bioreactor) that match the typical maximum split ratio to a production bioreactor (N bioreactor) from a 2,000 L (N-1) bioreactor.
Illustratively, a split ratio of 5:1 enables production bioreactor processing of 10,000 L with a 2,000 L seed reactor for the (N-1) stage.
4) Sterile connections between the stainless-steel bioreactors 18 and the single-use bioreactors 14 facilitate intensified fed-batch operations.
5) The majority of the stainless-steel bioreactors 18 are located in the stainless steel area 16 with lower air classification and lower architectural finishes, reducing building costs and operational costs (e.g., air purification costs).
6) The downstream purification area is reconfigurable to accommodate a broad range of outputs from the various upstream technologies, e.g., by utilizing a variety of readily movable production skids. The reconfigurable downstream purification area is configured to allow operations in batch mode or in connected mode, where batch mode involves pool vessels separating various purification steps while connected mode removes the pool vessels and employs direct connections between purification steps.
7) The reconfigurable downstream purification area is located adjacent to a reconfigurable seed train area within the reconfigurable, single-use area 12.
Each of the reconfigurable, single-use area 12 and the stainless steel area 16 may include, in some embodiments, subareas that are partially or wholly segregated (such as, e.g., by the partition 22, such as, e.g., a wall or plastic sheet) from other portions of the reconfigurable, single-use area 12 and the stainless steel area 16. Additionally, the reconfigurable, single-use area 12, in some embodiments, may include one or more pieces of equipment that are not single use (such as, e.g., a stainless steel centrifuge), so long as the piece of equipment is not a bioreactor. Similarly, the stainless steel area 16, in some embodiments, may include one or more pieces of equipment that are not made from stainless steel, so long as the piece of equipment is not a bioreactor.

The flexible facilities 10 disclosed herein are useful for manufacturing at least one therapeutic product (e.g., one; more than one (e.g., two, three, four, etc.) at a given time. Illustratively, the flexible facilities 10 are adapted for manufacturing a variety of different therapeutic products, either concurrently or sequentially, by deploying common resources where possible while facilitating interchangeability for process-specific equipment.

### Non-limiting Example Features

Without limitation, some example embodiments/features of the present disclosure include:
1. A flexible facility for manufacturing at least one therapeutic product, comprising:
   a reconfigurable, single-use area comprising at least one single-use bioreactor, wherein one or more of the at least one single use bioreactor(s) has a volume of at least 1000 L (e.g., 1000 L, 2000 L, 3000 L, 5000 L, at least 2000 L, at least 3000 L, at least 5000); and
   a stainless steel area comprising at least one stainless steel bioreactor, wherein each stainless steel bioreactor in the stainless steel area has a volume greater than 5000 L,
   wherein one or more of the at least one stainless steel bioreactor(s) is fluidly connected to one or more of the at least one single use bioreactor(s).
2. The flexible facility according to Feature 1, wherein one or more (e.g., one, two, three, four, five, six, seven, eight, nine, or ten) of the at least one single use bioreactor(s) has a volume of 1000 L, 2000 L, 3000 L, or 5000 L.
3. The flexible facility according to Feature 1 or 2, wherein one of the at least one single use bioreactor(s) has a volume of 1000 L, 2000 L, 3000 L, or 5000 L.
4. The flexible facility according to any one of Features 1 to 3, wherein no single-use bioreactor in the reconfigurable, single-use area has a volume greater than 2000 L.
5. The flexible facility according to any one of Features 1 to 4, wherein one or more (e.g., one, two, three, four, five, six, seven, eight, nine, or ten) of the at least one single use bioreactor(s) has a volume less than 2000 L (e.g., 1000 L, 500 L, 50 L).
6. The flexible facility according to any one of Features 1 to 5, wherein the reconfigurable, single-use area comprises one or more wave bioreactors (e.g., one or more wave bioreactors having a volume of at least 50 L (e.g., 50 L)) and/or one or more seed bioreactors (e.g. one or more seed bioreactors with a volume of at least 500 L (e.g., 500 L) or a volume of at least 2,000 L (e.g., 2,000 L)).
7. The flexible facility according to any one of Features 1 to 6, wherein the stainless steel area comprises a plurality of stainless steel bioreactors each having a volume of at least 10,000 L.
8. The flexible facility according to any one of Features 1 to 7, wherein each stainless steel bioreactor in the stainless steel area has a volume between 5000 L and 20,000 L.
9. The flexible facility according to any one of Features 1 to 8, wherein each stainless steel bioreactor in the stainless steel area has a volume between 5000 L and 15,000 L.
10. The flexible facility according to any one of Features 1 to 9, wherein each stainless steel bioreactor in the stainless steel area has a volume between 5000 L and 10,000 L.
11. The flexible facility according to any one of Features 1 to 10, wherein the stainless steel area comprises five 10,000 L bioreactors.
12. The flexible facility according to any one of Features 1 to 11, wherein one or more of the at least one stainless steel bioreactor(s) is supported on a ground surface.
13. The flexible facility according to any one of Features 1 to 12, wherein one or more of the at least one stainless steel bioreactor(s) is suspended from the structure itself.
14. The flexible facility according to any one of Features 1 to 13, wherein the reconfigurable, single-use area comprises a reagent preparation area, a seed train area, and a purification area.
15. The flexible facility according to any one of Features 1 to 14, wherein the reconfigurable, single-use area comprises a reagent preparation area, a seed train area, and a purification area, along with one or more partitions at least partially segregating one or more of the reagent preparation area, the seed train area, and the purification area.
16. The flexible facility according to Feature 14 or 15, wherein the seed train area comprises one or more pieces of equipment independently chosen from: tanks for storing cell culture media; tanks for storing acid, basic, and buffer solutions; inoculation preparation equipment; bioreactors (e.g., suitable for culturing cells), e.g., rocker bioreactors, 500 L bioreactors, and 2000 L single use bioreactors; tanks suitable for housing cells; and tanks suitable for housing products produced by cells.
17. The flexible facility according to any one of Features 14 to 16, wherein the equipment in the seed train area is in single-use format.
18. The flexible facility according to any one of Features 1 to 17, wherein the stainless steel area is adjacent to the seed train area.
19. The flexible facility according to any one of Features 1 to 17, wherein the stainless steel area is adjacent to the purification area.
20. The flexible facility according to any one of Features 1 to 17, wherein the stainless steel area is adjacent to both the seed train area and the purification area.
21. The flexible facility according to any one of Features 1 to 20, wherein the stainless steel area further comprises a harvest area.
22. The flexible facility according to any one of Features 1 to 21, wherein the stainless steel area is an unclassified manufacturing space.
23. The flexible facility according to any one of Features 1 to 22, wherein at least a portion of the reconfigurable, single-use area (e.g., a seed train area; a purification area) is a classified manufacturing space.
24. The flexible facility according to any one of Features 1 to 23, wherein the reconfigurable, single-use area and the stainless steel area are segregated by a partition.
25. The flexible facility according to Feature 24, wherein the partition is a plastic sheet.
26. The flexible facility according to Feature 25, wherein the partition is a wall.
27. The flexible facility according to Feature 26, wherein the stainless steel area is completely enclosed with separate air handling.
28. The flexible facility according to Feature 26 or 27, wherein a portion of the at least one stainless steel bioreactor(s) penetrates the wall and is fluidly connected through tubing to one or more of the at least one single use bioreactor(s).
29. The flexible facility according to any one of Features 26 to 28, wherein at least 5% of the exterior vessel surface area of the at least one stainless steel bioreactor(s) penetrates the wall and is fluidly connected through tubing to at least one of the at least one single use bioreactor(s).
30. The flexible facility according to Feature 28 or 29, wherein the portion penetrating the wall comprises one or more components independently chosen from probe belts to allow access to bioreactor instrumentation, connections for perfusion, and connections for feed solutions.
31. The flexible facility according to any one of Features 1 to 30, wherein the fluid connection comprises an aseptic connecting module.
32. The flexible facility according to any one of Features 1 to 31, wherein an aseptic connecting module connects one or more seed train bioreactors in the reconfigurable, single-use area to one or more stainless steel bioreactors in the stainless steel area.
33. The flexible facility according to any one of Features 1 to 32, wherein the fluid connection is configured to enable bypass of the stainless steel area.
34. The flexible facility according to any one of Features 1 to 33, wherein the fluid connection is configured to enable bypass of the stainless steel area through the use of one or more valves.
35. The flexible facility according to any one of Features 1 to 34, wherein the fluid connection is removable.
36. The flexible facility according to any one of Features 1 to 35, wherein the fluid connection is capable of being moved between different bioreactors within the flexible facility.
37. The flexible facility according to any one of Features 1 to 36, wherein the flexible facility comprises a plurality of connections capable of being reconfigured to connect different equipment (e.g., bioreactors, purification skids, etc.) in the flexible facility.
38. The flexible facility according to any one of Features 1 to 37, wherein the reconfigurable, single-use area and/or the stainless steel area further comprises one or more centrifuges.
39. The flexible facility according to any one of Features 1 to 38, wherein the reconfigurable, single-use area comprises one or more seed trains.
40. The flexible facility according to any one of Features 1 to 39, wherein the reconfigurable, single-use area and/or the stainless steel area comprises one or more purification skids (e.g., one or more purification skids that contain only single-use equipment; one or more purification skids that contain only stainless steel equipment; one or more purification skids that contain single-use equipment and stainless steel equipment).
41. The flexible facility according to Feature 40, wherein each of the one or more purification skids comprises one or more components independently chosen from chromatography skids, chromatography columns, viral inactivation systems, viral filtration systems, ultrafiltration diafiltration systems, and bulk filtration systems.
42. The flexible facility according to any one of Features 1 to 41, further comprising one or more additional spaces independently chosen from utility spaces, media buffer facilities, personnel changing rooms, and production units.
43. The flexible facility according to any one of Features 1 to 42, wherein the flexible facility is configured for producing one or more drug substances independently chosen from monoclonal antibodies, BiTE® molecules, RNA-based drug substances, and fermentation products.
44. The flexible facility according to any one of Features 1 to 43, wherein the flexible facility is configured to implement one or more processes independently chosen from mammalian cell culture fed-batch processes, mammalian cell culture perfusion processes, continuous mammalian cell culture processes, microbial fermentation fed-batch processes, and combinations of any of the foregoing.
45. The flexible facility according to any one of Features 1 to 44, wherein the flexible facility may be constructed in a series of phases.
46. The flexible facility according to any one of Features 1 to 45, wherein the flexible facility is capable of being expanded to add additional product lines.
47. The flexible facility according to any one of Features 1 to 46, wherein the stainless steel bioreactor(s) in the stainless steel area contain(s) only one therapeutic product-producing cell line.
48. The flexible facility according to any one of Features 1 to 46, wherein the stainless steel bioreactor(s) in the stainless steel area contain(s) more than one therapeutic product-producing cell line.
49. The flexible facility according to any one of Features 1 to 48, wherein the single-use bioreactor(s) in the reconfigurable, single-use area contain(s) only one therapeutic product-producing cell line.
50. The flexible facility according to any one of Features 1 to 48, wherein the single-use bioreactor(s) in the reconfigurable, single-use area contain(s) more than one therapeutic product-producing cell line.
51. The flexible facility according to any one of Features 47 to 50, wherein each therapeutic product-producing cell line is independently chosen from prokaryotic cell lines and eukaryotic cell lines.
52. The flexible facility according to any one of Features 47 to 50, wherein each therapeutic product-producing cell line is independently chosen from prokaryotic cell lines.
53. The flexible facility according to any one of Features 47 to 50, wherein each therapeutic product-producing cell line is independently chosen from mammalian cell lines.
54. The flexible facility according to any one of Features 47 to 50, wherein each therapeutic product-producing cell line is independently chosen from cell lines capable of producing a viral therapeutic (e.g., an anti-cancer oncolytic virus, a viral vector for gene therapy and/or viral immunotherapy).
55. The flexible facility according to any one of Features 47 to 50, wherein each therapeutic product-producing cell line is independently chosen from CHO cell lines.

### DEFINITIONS

As used herein, the terms “a” and “an” mean “one or more” unless specifically indicated otherwise. Additionally, “one or more” and “at least one” are used interchangeably herein. Furthermore, unless otherwise required by context, singular terms include pluralities and plural terms include the singular.

As used herein, the term “bioreactor” means any vessel useful for the growth of a cell culture (e.g., a mammalian cell culture or a bacterial cell culture). “Bioreactor” encompasses the term “fermenter” (i.e., a vessel useful for the growth of a bacterial cell culture, which typically contains a more rigorous agitator and increased gas flow relative to a vessel used for the growth of a mammalian cell culture) herein. Non-limiting examples of bioreactors include stirred tank, airlift, fiber, microfiber, hollow fiber, ceramic matrix, fluidized bed, fixed bed, and/or spouted bed bioreactors. In some embodiments, an example bioreactor can perform one or more (e.g., one, two, three, all) of the following steps: feeding of nutrients and/or carbon sources, injection of suitable gas (such as, e.g., oxygen), inlet and outlet flow of fermentation or cell culture medium, separation of gas and liquid phases, maintenance of temperature, maintenance of oxygen and CO2 levels, maintenance of pH level, agitation (e.g., stirring), and/or cleaning/sterilizing. Unless otherwise indicated by context, a bioreactor can be suitable for batch, semi fed-batch, fed-batch, perfusion, and/or continuous fermentation processes. Any suitable bioreactor diameter can be used. Unless otherwise indicated by context, in some embodiments, the bioreactor can have a volume between 100 mL and 50,000 L. Unless otherwise indicated, a bioreactor can be of any size so long as it is useful for the culturing of cells; typically, a bioreactor is sized appropriate to the volume of cell culture being grown inside of it. In non-limiting embodiments and unless otherwise indicated by context, a bioreactor may be at least 1 liter (L) or may be 2, 5, 10, 50, 100, 200, 250, 500, 1,000, 1,500, 2,000, 2,500, 5,000, 8,000, 10,000, 12,000 liters or more, or any volume in between. The internal conditions of the bioreactor, including, but not limited to, pH and temperature, can be controlled during the culturing period. Those of ordinary skill in the art will be aware of, and will be able to select, suitable bioreactors for use in the flexible facilities disclosed herein based on the relevant considerations.

As used herein, the term “cell culture” or “culture” refers to the growth and propagation of cells outside of a multicellular organism or tissue. Suitable culture conditions for mammalian and bacterial cells are known in the art. (See, e.g., Animal cell culture: A Practical Approach, D. Rickwood, ed., Oxford University Press, New York (1992).) Mammalian cells may be cultured in suspension or while attached to a solid substrate. In some embodiments, fluidized bed bioreactors, hollow fiber bioreactors, roller bottles, shake flasks, and/or stirred tank bioreactors, with or without microcarriers, may be used for cell culture. In some embodiments, 500 L to 2000 L bioreactors are used for cell culture (e.g., as part of a seed train). In some embodiments, 1000 L to 2000 L bioreactors are used for cell culture (e.g., as part of a seed train).

As used herein, the term “cell culturing medium” (also referred to as “media,” “culture medium,” “cell culture media,” “tissue culture media,” and the like) refers to any nutrient solution used for growing cells, e.g., bacterial or mammalian cells. Cell culturing medium generally provides one or more of the following components: an energy source (e.g., in the form of a carbohydrate, such as, e.g., glucose); one or more essential amino acids (e.g., all essential amino acids; the twenty basic amino acids plus cysteine); vitamins and/or other organic compounds typically required at low concentrations; lipids or free fatty acids; and trace elements, such as, e.g., inorganic compounds or naturally occurring elements that are typically required at very low concentrations, such as, e.g., concentrations in the micromolar range. As used herein, cell culturing medium encompasses nutrient solutions that are typically employed in and/or are known for use with any cell culture process, including, but not limited to, batch, extended batch, fed-batch, and/or perfusion or continuous culturing of cells.

As used herein, “fed-batch culture” refers to a form of suspension culture, specifically a method of culturing cells in which additional components are provided to the culture at a time or times subsequent to the beginning of the culture process. The provided components typically comprise nutritional supplements for the cells which have been depleted during the culturing process. Additionally or alternatively, the additional components may include supplementary components (such as, e.g., a cell-cycle inhibitory compound). In some embodiments, fed-batch cell culture medium formulations may be richer or more concentrated than base cell culture medium formulations. A fed-batch culture may be stopped at some point, and the cells and/or components in the medium may be harvested and optionally purified.

As used herein, a “perfusion” cell culture medium refers to a cell culture medium that is typically used in cell cultures that are maintained by perfusion or continuous culture methods and is sufficiently complete to support the cell culture during this process. In some embodiments, perfusion cell culture medium formulations may be richer or more concentrated than base cell culture medium formulations to accommodate the method used to remove the spent medium. In some embodiments, perfusion cell culture medium may be used during both the growth and production phases.

As used herein, a “production” cell culture medium refers to a cell culture medium that is typically used in a cell culture during the transition when exponential growth is ending and protein production takes over (i.e., “transition” and/or “product” phases) and is sufficiently complete to maintain a desired cell density, viability, and/or product titer during this phase.

As used herein, a “therapeutic product” is a biological product for use in a subject (e.g., a human) in connection with preventing, diagnosing, curing, or alleviating a disease, ailment, defect, or injury. Non-limiting examples of therapeutic products include monoclonal antibodies, BiTE® molecules, RNA-based drug substances, cell therapies, and fermentation products.

### Seed Train Area

A seed train area, such as an area of the single-use area 12 in which the one or more seed trains 34 are disposed, in the flexible facility 10 may house equipment suitable for cell culture. Equipment for cell culture includes, but is not limited to: tanks for storing cell culture media; tanks for acidic, basic, and buffer solutions; inoculation preparation; bioreactors (e.g., suitable for culturing cells), including rocker bioreactors, 500 L bioreactors, and 2000 L single-use bioreactors, such as the single use bioreactors 14; tanks suitable for housing cells or products produced by cells; centrifuges, such as the centrifuges 32; pumps; and other equipment useful for product harvest. In some embodiments, the equipment in the seed train area is in single use format.

In some embodiments, the seed train area demonstrates the flexibility of the manufacturing facilities of the present disclosure. For example, in some configurations where stainless steel bioreactors 18 are also employed, the seed train may contain bioreactor units up to and including the N-1 bioreactor. In some embodiments, the product of the N-1 bioreactor is transferred to the stainless steel N bioreactor. In this configuration, equipment for product harvest is bypassed in favor of the equipment in the stainless steel area.

In some configurations where the stainless steel section is bypassed, the largest bioreactor units are considered the N bioreactors (such as, e.g., 500 L or 2000 L N bioreactors) and the equipment in the seed train area for product harvest is utilized.

In some embodiments, the seed train area contains one or more flasks, culture bags, and bioreactor units suitable for culturing cells. In some embodiments, the cell culture units in the seed train area become progressively larger within a seed train to accommodate larger cell cultures. In some embodiments, a seed train may comprise one wave bioreactor and two seed bioreactors. In some embodiments, a seed train may comprise one 50 L wave bioreactor, one 500 L single-use bioreactor, and one 2000 L single-use bioreactor, wherein the 2000 L single-use bioreactor, such as the single-use bioreactor 14, is optionally configured for perfusion culture (such as, e.g., ATF perfusion culture). In some embodiments, there may also be parallel sets of progressively larger bioreactors, wherein each parallel set may contain cells expressing the same product or may contain cells expressing a different product.

In some embodiments, the seed train area may contain 1, 2, 3, 4, 5, 6, 7, 8, 9, or 10 bioreactors, or multiples thereof in the case of the manufacture of multiple products. In some embodiments, each bioreactor in the seed train area is suitable for one or more process methods independently chosen from batch, semi fed-batch, fed-batch, perfusion, and/or continuous processes. In some embodiments, the bioreactor is a stirred tank reactor. In some embodiments, the bioreactor is an airlift reactor. In some embodiments, the bioreactor can have a volume between about 100 milliliters and about 5,000 liters. Non-limiting example bioreactor volumes include 100 milliliters, 250 milliliters, 500 milliliters, 750 milliliters, 1 liter, 2 liters, 3 liters, 4 liters, 5 liters, 6 liters, 7 liters, 8 liters, 9 liters, 10 liters, 15 liters, 20 liters, 25 liters, 30 liters, 40 liters, 50 liters, 60 liters, 70 liters, 80 liters, 90 liters, 100 liters, 150 liters, 200 liters, 250 liters, 300 liters, 350 liters, 400 liters, 450 liters, 500 liters, 550 liters, 600 liters, 650 liters, 700 liters, 750 liters, 800 liters, 850 liters, 900 liters, 950 liters, 1000 liters, 1500 liters, 2000 liters, 2500 liters, 3000 liters, 3500 liters, 4000 liters, 4500 liters, and 5000 liters. Generally, the bioreactor units in the seed train area are smaller than the bioreactor units in the stainless steel area. In certain configurations, each seed train bioreactor is smaller than the stainless steel production bioreactor by a factor of at least 2, at least 3, at least 4, or at least 5. In some embodiments, the seed train section can house four 2,000 liter vessels for single-use technology operations.

In some embodiments, the seed train area is in compliance with good manufacturing process and biological safety standards. In some embodiments, the seed train area is compliant with biosafety level 1 (BSL1), biosafety level 2 (BSL2), biosafety level 3 (BSL3), or biosafety level 4 (BSL4) standards.

In some embodiments, the seed train area can comprise sub-compartments in which each sub-compartment can be used to perform a different function or aspect that supports the cell culture production processes. By way of example, in some embodiments, the seed train area can comprise a sub-compartment that houses one or more bioreactors and a sub-compartment that houses equipment for product harvest.

### Single-use Production Area

In some embodiments wherein the stainless steel area 16 is bypassed, the reconfigurable, single-use area 12 comprises one or more production bioreactors. The flexible facility 10 is easily expandable and scalable, and different production bioreactors within the reconfigurable, single-use area 12 can be used to produce completely different therapeutic products in the same flexible facility 10.

For example, in some embodiments, in a modular unit configured as a first bioreactor module, a user could be manufacturing one type of therapeutic product, such as, e.g., a monoclonal antibody product derived from a mammalian cell line. In a second modular unit, the user could manufacture a completely different therapeutic product, such as, e.g., a microbial product. In some embodiments, the flexible facility 10 of the present disclosure is capable of supporting multiple product lines simultaneously, using production bioreactors in one or both of the single-use production area 12 and the stainless steel area 16. Moreover, in some embodiments, a flexible facility, such as the flexible facility 10, of the present disclosure is capable of being expanded to add additional product lines.

### Stainless Steel Area

In some embodiments, the stainless steel section 16 houses equipment suitable for cell culture. Equipment suitable for cell culture includes, but is not limited to, bioreactors (e.g., suitable for culturing cells), tanks (e.g., suitable for housing cells, media or products produced by cells), centrifuges, pumps, and other equipment useful for product harvest, as well as a cleaning system (CIP). A CIP unit is typically a modular skid and has several tanks to hold a cleaning solution (such as, e.g., a caustic solution and/or bleach), pumps, and sensors to send the cleaning solution to the appropriate tank to be cleaned.

In some embodiments, the stainless steel section 16 contains one or more stainless steel bioreactor units, such as the stainless steel bioreactor(s) 18, suitable for culturing cells. A bioreactor unit can perform one or more (such as, e.g., one, two, three, four, all) of the following: feeding of nutrients and/or carbon sources; injection of suitable gas (e.g., oxygen); flow of cell culture medium; separation of gas and liquid phases; maintenance of growth temperature; maintenance of pH level; agitation (e.g., stirring); and/or cleaning/sterilizing. In some embodiments, the stainless steel section may contain 1, 2, 3, 4, 5, 10, 15, 20, 25, 30, 35, 40, 45, 50, 60, 70, 80, 90, or 100 or more bioreactors. In some embodiments, each bioreactor in the stainless steel area 16 is suitable for one or more process methods independently chosen from batch, semi fed-batch, fed-batch, perfusion, and/or continuous processes. In some embodiments, at least one stainless steel bioreactor 18 is a stirred tank reactor. In some embodiments, at least one stainless steel bioreactor 18 is an airlift reactor. In some embodiments, at least one stainless steel bioreactor 18 can have a volume between 1000 liters and 50,000 liters. Non-limiting examples include a volume of 1000 liters, 1500 liters, 2000 liters, 2500 liters, 3000 liters, 3500 liters, 4000 liters, 4500 liters, 5000 liters, 6000 liters, 7000 liters, 8000 liters, 9000 liters, 10,000 liters, 15,000 liters, 20,000 liters, or 50,000 liters.

In some embodiments, approximately 95% of the stainless steel bioreactor(s) reside in a non-classified space within the flexible facility 10 with a small portion of the stainless steel bioreactor(s) 18 (approximately 5%) exposed to the classified reconfigurable, single-use area 12 of the flexible facility 10. In some embodiments, the exposed portion(s) of the stainless steel bioreactor(s) 18 may include the probe belt 27a of the tank to allow access to tank instrumentation, connections 27b for perfusion, and any other connections 27c for feeds and adding solutions to the tanks, including connections for pH control, nutrient feeds (media), or other additions (i.e., anti-foam addition).

In some embodiments, the exposed portion(s) 24 of the stainless steel bioreactor(s) 18 is/are directly exposed into the classified space, such as, e.g., with the probe belt 27a, or through one or more connections made to one or more lines in the classified space that may enter the tank through piping in other tank locations (such as, e.g., with the media additions).

In some embodiments, the stainless steel area 16 is designed to accommodate a number of bioreactors 18. In some embodiments, the stainless steel area 16 can house five 10,000 liter vessels for a mammalian cell line.

In some embodiments, the stainless steel area 16 comprises five or more 10,000 liter vessels and harvesting equipment. In some embodiments, the stainless steel area 16 comprises five 10,000 liter vessels and harvesting equipment. In some embodiments, the bioreactors in the stainless steel area are configured for manufacturing a monoclonal antibody product derived from a mammalian cell line.

In some embodiments, one or more stainless steel bioreactors 18 in the stainless steel area 16 may be ground-based reactors. In some embodiments, one or more stainless steel bioreactors 18 may be suspended from the structure itself.

In some embodiments, the stainless steel area 16 is in compliance with good manufacturing process and biological safety standards. In some embodiments, the stainless steel area 16 is compliant with biosafety level 1 (BSL1), biosafety level 2 (BSL2), biosafety level 3 (BSL3), or biosafety level 4 (BSL4) standards.

In some embodiments, the stainless steel area 16 may comprise sub-compartments in which each sub-compartment can be used to perform a different function or aspect that supports the cell culture production processes. By way of example, in some embodiments, the stainless steel area 16 may comprise a sub-compartment that houses one or more bioreactors, a sub-compartment that houses equipment for product harvest, a subcompartment for inoculum, and a sub-compartment for cleaning and decontamination of equipment and the operators handling such equipment.

In some embodiments, different production bioreactors within the stainless steel area 16 can be used to produce completely different therapeutic products in the same flexible facility, such as the flexible facility 10. For example, in some embodiments, in a modular unit configured as a first bioreactor module, a user could be manufacturing one type of therapeutic product, such as, e.g., a monoclonal antibody product derived from a mammalian cell line. In a second modular unit, the user could manufacture a completely different therapeutic product, such as, e.g., a microbial product.

### Downstream Processing Area

A standard downstream processing (DSP) unit is generally an assembly of chromatography, mixing, and filtration equipment. In non-limiting embodiments of the disclosure, filtration equipment in the downstream processing area can include ultrafiltration, microfiltration, viral filtration, and sterilization equipment, including, e.g., pre-viral separation and post-viral separation sub-units. Viral reduction may occur throughout a typical mammalian cell derived protein purification process; for example, in some embodiments, viral reduction may occur through a viral reducing chromatography step(s), viral inactivation with mixed vessels, and/or viral filtration-all within one mammalian process. A post-viral separation sub-unit houses equipment and utilities suitable for any one of the following: ultrafiltration (tangential filtration); normal filtration; chromatography; formulation; titration; mixing; concentration; buffer exchange; and bulk drug substance container filling and freezing.

A non-limiting example purification area may include: a tank with a stirrer to which there is connected an inlet conduit of the crude extracted product from the separation module; an outlet conduit, provided with a volumetric pump, connected to an inlet conduit in a first chromatography column run by a computerized unit to which the conduit is also connected; an outlet conduit from the chromatography column connected to an inlet conduit in a tank provided with a stirrer, an outlet conduit of which, provided with a volumetric pump, is connected to an inlet conduit in a second chromatography column run by a computerized unit to which the conduit is also connected; an outlet conduit from the chromatography column connected to an inlet conduit in a tank provided with a stirrer, an outlet conduit of which is in turn connected in a discontinuous manner to the inlet of a vacuum-freeze dryer, provided with an outlet.

### Classification Levels

Different areas within the flexible facility 10 (such as, e.g., the reconfigurable, single-use area 12 and the stainless steel area 16, as well as portions of the same) can have different classification levels based on grading standards. For example, in some embodiments, different areas can have different classification levels based on grading standards set by a regulatory body in a jurisdiction in which the flexible facility 10 is regulated. In some embodiments, different areas can have different classification levels based on grading standards set by a regulatory body in a jurisdiction in which the flexible facility 10 is located. In some embodiments, different areas can have different classification levels based on grading standards set by a regulatory body in a jurisdiction in which a therapeutic product produced in the flexible facility 10 is sold.

In some embodiments, different areas can have different classification levels based on grading standards set by the United States Food and Drug Administration or grading standards set by EudraLex, the Rules Governing Medicinal Products in the European Union Volume 4 EU Guidelines to Good Manufacturing Practice Medicinal Products for Human and Veterinary Use, supplemented by Annex 1 Manufacture of Sterile Medicinal Products in the European Union.

### Control Systems

In some embodiments, samples from a cell culture can be monitored and evaluated using any of the analytical techniques known in the art. In some embodiments, a variety of parameters including, but not limited to, recombinant protein and medium quality and characteristics can be monitored for the duration of the culture. Samples can be taken and monitored intermittently at a desirable frequency, including continuous monitoring, real time monitoring, or near real time monitoring.

In some embodiments, the seed train equipment, stainless steel bioreactors, and purification equipment may each include sensors for monitoring reaction parameters and/or product quality parameters. The parameters monitored can include, but are not limited to, conductivity, temperature, pH, oxygen, and CO₂. The sensors may be any type of invasive sensor known in the art for monitoring these parameters, where the sensors are in contact with a process fluid. In addition, in some embodiments, the sensors may be non-invasive optical chemical sensors, such as those described, e.g., in U.S. Pat. Nos. 6,673,532 and 7,041,493, and U.S. Patent Application Publication No. 20110065084. In addition, spectrometers known in the art can be used in the seed train equipment, production bioreactors, and/or the purification equipment to monitor the product stream and/or the inputs to each equipment. The parameters measured by such spectrometers can include, but are not limited to, absorbance, multi-angle light scattering, fluorescence, Raman scattering, circular dichroism, and infrared spectral characteristics.

In some embodiments, the equipment and processes within each area are computer controlled and monitored via a computer system and corresponding software, which also allows automation of the system. The equipment located in each area may include data collection sensors and controls which may be in communication with a central controller either via hardwire or wireless connection.

In some embodiments, each area may additionally include an electronics control panel, which may also be used to monitor, collect, and control the equipment and processes occurring therein. The central controller may also include data storage to store data, as well as application and/or operation software for the central controller and system operation. The software may include a graphical-user-interface (GUI) displayable on a workstation. The GUI may display a number of different screens for setting up, monitoring, and controlling the equipment and the overall manufacturing process.

Accordingly, in some embodiments, such software may be used to control a procedure within an area and optionally may be customized via the central computer and/or an electronic control panel. A procedure is a strategy for carrying out a particular process in an area (i.e., material produced or that has been produced by a single execution of a batch process). In some embodiments, a process may be a sequence of chemical, physical, or biological activities for the conversion, transport, or storage of material or energy.

In some embodiments, an aseptic connecting module connects one or more seed train bioreactors in the reconfigurable, single-use area to one or more stainless steel bioreactors in the stainless steel area.

In some embodiments, an aseptic connecting module connects a harvesting unit to one or more downstream purification units.

### Cell Lines

In some embodiments, each bioreactor in a flexible facility 10 of the present disclosure is suitable for culturing prokaryotic cells or eukaryotic cells. In some embodiments, at least one bioreactor in the flexible facility 10 is suitable for culturing prokaryotic cells. In some embodiments, at least one bioreactor in the flexible facility 10 is suitable for culturing eukaryotic (such as, e.g., mammalian) cells. In some embodiments, at least one bioreactor in the flexible facility 10 is suitable for culturing cells capable of producing a viral therapeutic (e.g., an anti-cancer oncolytic virus, a viral vector for gene therapy, and/or viral immunotherapy). In some embodiments, at least one bioreactor in the flexible facility 10 is suitable for culturing prokaryotic cells capable of producing a viral therapeutic (e.g., an anti-cancer oncolytic virus, a viral vector for gene therapy, and/or viral immunotherapy). In some embodiments, at least one bioreactor in the flexible facility 10 is suitable for culturing eukaryotic (such as, e.g., mammalian) cells capable of producing a viral therapeutic (e.g., an anti-cancer oncolytic virus, a viral vector for gene therapy and/or viral immunotherapy).

Examples of suitable cells for culture in the bioreactors of the flexible facilities 10 of the present disclosure include, but are not limited to, bacterial cells (e.g., E. coli, P. pastoris), yeast cells (e.g., S. cerevisae, T. reesei), plant cells, insect cells (e.g., Sf9), Chinese hamster ovary cells (CHO, and any genetically modified or derived CHO cell line), mouse cells (e.g., mouse embryonic fibroblasts, cells derived from mouse cancer models), human cells (e.g., cells from any tissue or organ, cells from a cancer or other diseased cell line, stem cells hybridoma cells, or other genetically modified or hybrid cells. Suitable host cells for use in a bioreactor contained in a flexible facility of the present disclosure include those that are commercially available, for example, from culture collections such as the DSMZ (Deutsche Sammlung von Mikroorganismen and Zellkulturen GmbH, Braunschweig, Germany) or the American Type Culture Collection (ATCC).

In some embodiments, the cells are bacterial or prokaryotic cells. In some embodiments, the prokaryotic cells are chosen from Gram positive cells, such as, e.g., Bacillus (such as, e.g., B. subtilis (such as, e.g., B. subtilis 3NA and B. subtilis 168), B. amyloliquefaciens, B. licheniformis, B. natto, or B. megaterium), Streptomyces, Streptococcus, Staphylococcus, or Lactobacillus cells. In some embodiments, the prokaryotic cells are chosen from Gram negative cells, such as, e.g., Salmonella spp. or Escherichia coli, such as, e.g., TGI, TG2, W3110, DH1, DHB 4, DH5a, HMS 174, HMS174 (DE3), NM533, C600, HB101, JM109, MC4100, XLI-Blue, and Origami cells, as well as cells derived from E. coli B-strains, such as, e.g., BL- 21 or BL 21 (DE3). Prokaryotic or bacterial cells may be obtained from commercial vendors. Bacillus cells, for example, are obtainable from, for example, the Bacillus Genetic Stock Center, Biological Sciences 556, 484 West 12tA Avenue, Columbus Ohio 43210-1214.

In some embodiments, the cells are eukaryotic cells, such as, e.g., mammalian cells. The mammalian cells can be, for example, human or rodent or bovine cell lines or cell strains. Examples of such cells, cell lines, or cell strains include, but are not limited to, mouse myeloma (NSO)-cell lines, Chinese hamster ovary (CHO)-cell lines, FIT 1080, H9, HepG2, MCF7, MDBK Jurkat, NIH3T3, PC12, BF1K (baby hamster kidney cell), VERO, SP2/0, YB2/0, Y0, C127, L cell, COS, e.g., COS1 and COS7, QC1-3, HEK-293, VERO, PER. C6, HeLa, EB1, EB2, EB3, oncolytic, or hybridoma-cell lines. In some embodiments, the mammalian cells are CHO-cell lines. In some embodiments, the mammalian cells are CHO cells. In some embodiments, the mammalian cells are chosen from CHO-K1 cells, CHO-K1 SV cells, DG44 CHO cells, DUXB11 CHO cells, CHOS cells, CHO GS knock-out cells, CHO FUT8 GS knock-out cells, CHOZN cells, and CHO derived cells. A CHO GS knock-out cell (such as, e.g., a GSKO cell) is, for example, a CHO-K1 SV GS knockout cell. Additionally, the CHO FUT8 knockout cell is, for example, the Potelligent® CHOK1 SV (Lonza, Inc.). In some embodiments, the eukaryotic cells can also be avian cells, cell lines, or cell strains, such as, e.g., EBx® cells, EB14, EB24, EB26, EB66, or EBv13.

In some embodiments, any mammalian cell line can be used in a bioreactor included in a flexible facility 10 of the present disclosure. As non-limiting examples, a wide variety of mammalian cell lines suitable for growth in culture are available from the American Type Culture Collection (Manassas, Va.) and commercial vendors. Non-limiting examples of cell lines commonly used in the industry include monkey kidney CVI line transformed by SV 40 (COS-7, ATCC CRL 1651); human embryonic kidney line (293 or 293 cells subcloned for growth in suspension culture, (Graham et al, 1977, J. Gen Virol. 36:59); baby hamster kidney cells (BHK, ATCC CCL 10); mouse Sertoli cells (TM4, Mather, 1980, Biol. Reprod. 23:243-251); monkey kidney cells (CVI ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human hepatoma cells (Hep G2, HB 8065); mouse mammary tumor (MMT 060562, ATCC CCL 51); TRI cells (Mather et al., 1982, Annals N. Y Acad. Sci. 383:44-68); MRC 5 cells or FS 4 cells; mammalian myeloma cells, and a number of other cell lines and Chinese hamster ovary (CHO) cells.

CHO cells, including CHOK1 cells (ATCC CCL61), are widely used to produce complex recombinant proteins. In some embodiments, the dihydrofolate reductase (DHFR)-deficient mutant cell lines (Urlaub et al., 1980, Proc Natl Acad Sci USA 77: 4216-4220), DXB11 and DG-44, are desirable CHO host cell lines because the efficient DHFR selectable and amplifiable gene expression system allows high level recombinant protein expression in these cell lines (Kaufman R. J., 1990, Meth Enzymol 185:537-566). Also included are the glutamine synthase (GS)-knockout CHOK1SV cell lines, making use of glutamine synthetase (GS)-based methionine sulfoximine (MSX) selection. Other suitable CHO host cells for use in a bioreactor of a flexible facility of the present disclosure include, but are not limited to, the following (ECACC accession numbers in parenthesis): CHO (85050302); CHO (PROTEIN1 FREE) (00102307); CHO-K1 (85051005); CHO-K1/SF (93061607); CHO/dhFr-(94060607); Cho/dhfr-ac-free (05011002); and Rr-choki (92052129).

Large-scale production of proteins for commercial applications may be carried out in suspension culture. Therefore, mammalian host cells used to generate the recombinant mammalian cells described herein can, but need not be, adapted to growth in suspension culture. A variety of host cells adapted to growth in suspension culture are known, including mouse myeloma NSO cells and CLIO cells from CFIO-S, DG44, and DXB11 cell lines. Other suitable cell lines include, but are not limited to, mouse myeloma SP2/0 cells, baby hamster kidney BF1K-21 cells, human PER. C6® cells, human embryonic kidney F1EK-293 cells, and cell lines derived or engineered from any of the cell lines disclosed herein.

In some embodiments, the eukaryotic cells are chosen from lower eukaryotic cells, such as, e.g., yeast cells (e.g., Pichia genus (e.g., Pichia pastoris, Pichia methanolica, Pichia kluyveri, and Pichia angusta), Komagataella genus (e.g., Komagataella pastoris, Komagataella pseudopastoris, or Komagataella phaffii), cells of the Saccharomyces genus (e.g., Saccharomyces cerevisae, Saccharomyces kluyveri, Saccharomyces uvarum), cells of the Kluyveromyces genus (e.g., Kluyveromyces lactis, Kluyveromyces marxianus), cells of the Candida genus (e.g., Candida utilis, Candida cacaoi, Candida boidinii), cells of the Geotrichum genus (e.g., Geotrichum fermentans), Haη-senula polymorpha, Yarrowia lipolytica, or Schizosaccharomyces pombe. In some embodiments, the eukaryotic cells are chosen from Pichia pastoris strains. Non-limiting examples of Pichia pastoris strains include X33, GS115, KM71, KM71H, and CBS7435.

In some embodiments, the eukaryotic cells are chosen from fungal cells (e.g., cells of Aspergillus (such as, e.g., A. niger, A. fumigatus, A. orzyae, A. nidula),Acremonium (such as, e.g., A. thermophilum), Chaetomium (such as, e.g., C. thermophilum), Chrysosporium (such as, e.g., C. thermophile), Cordyceps (such as, e.g., C. militaris), Corynascus, Ctenomyces, Fusarium (such as, e.g., F. oxysporum), Glomerella (such as, e.g., G. graminicola), Hypocrea (such as, e.g., H. jecorina), Magnaporthe (such as, e.g., M. orzyae), Myceliophthora (such as, e.g., M. thermophile), Nectria (such as, e.g., N. heamatococca), Neurospora (such as, e.g., N. crassa), Penicillium, Sporotrichum (such as, e.g., S. thermophile), Thielavia (such as, e.g., T. terrestris, T. heterothallica), Trichoderma (such as, e.g., T. reesei), or Verticillium (such as, e.g., V. dahlia).

In some embodiments, the eukaryotic cells are chosen from insect cells (such as, e.g., Sf9, Mimic™ Sf9, Sf21, High Five ™M (BT1-TN-5B1-4), or BT1-Ea88 cells), algae cells (such as, e.g., of the genus Amphora, Bacillariophyceae, Dunaliella, Chlorella, Chlamydomonas, Cyanophyta (cyanobacteria), Nannochloropsis, Spirulina, or Ochromonas), and plant cells (such as, e.g., cells from monocotyledonous plants (such as, e.g., maize, rice, wheat, or Setaria), or cells from a dicotyledonous plants (such as, e.g., cassava, potato, soybean, tomato, tobacco, alfalfa, Physcomitrella patens or Arabidopsis)).

In some embodiments, the cells express or produce a product, such as, e.g., a recombinant therapeutic product. As described in more detail below, examples of therapeutic products produced by cells include, but are not limited to, antibody molecules (e.g., monoclonal antibodies, bispecific antibodies), antibody mimetics (polypeptide molecules that bind specifically to antigens but that are not structurally related to antibodies, such as, e.g., DARPins, afiSbodies, adnectins, or IgNARs), fusion proteins (e.g., Fc fusion proteins, chimeric cytokines), other recombinant proteins (e.g., glycosylated proteins, enzymes, hormones), viral therapeutics (e.g., anti-cancer oncolytic viruses, viral vectors for gene therapy and viral immunotherapy), cell therapeutics (e.g., pluripotent stem cells, mesenchymal stem cells, and adult stem cells), vaccines or lipid-encapsulated particles (e.g., exosomes, virus-like particles), RNA (e.g., siRNA) or DNA (e.g., plasmid DNA), antibiotics, or amino acids. In some embodiments, a flexible facility according to the present disclosure can be used for producing biosimilars. In some embodiments, a flexible facility according to the present disclosure can be used for producing therapeutic products with a range of complexity, which may include posttranslational modifications, such as, e.g., glycosylation.

In some embodiments, a flexible facility according to the present disclosure allows for the production of eukaryotic cells, e.g., mammalian cells or lower eukaryotic cells, such as, e.g., yeast cells or filamentous fungi cells, or prokaryotic cells, such as, e.g., Gram-positive or Gram-negative cells and/or products of the eukaryotic or prokaryotic cells, e.g., proteins, peptides, antibiotics, amino acids, nucleic acids (such as, e.g., DNA or RNA), synthesized by the eukaryotic cells in a large-scale manner.

### Culture Methods

Various methods that may used in commercial processes for the production of recombinant proteins by mammalian cell culture include, but are not limited to: batch culture; fed-batch culture; and perfusion culture.

Batch culture is a discontinuous method where cells are grown in a fixed volume of culture media for a short period of time followed by a full harvest. Cultures grown using the batch method experience an increase in cell density until a maximum cell density is reached, followed by a decline in viable cell density as the media components are consumed and levels of metabolic by-products (such as lactate and ammonia) accumulate. Harvest typically occurs at the point when the maximum cell density is achieved (e.g., 5×10⁶ cells/mL or greater, depending on media formulation, cell line, etc.). The batch process is the simplest culture method; however, viable cell density is limited by nutrient availability and once the cells are at maximum density, the culture declines and production decreases. There is no ability to extend a production phase in batch culture because the accumulation of waste products and nutrient depletion rapidly lead to culture decline, typically around 3 to 7 days.

Fed-batch culture improves on the batch process by providing bolus or continuous media feeds to replenish those media components that have been consumed. Since fed-batch cultures receive additional nutrients throughout the run, they have the potential to achieve higher cell densities (>10 to 30×10⁶ cells/mL, depending on media formulation, cell line, etc.) and increased product titers, when compared to the batch method. Unlike the batch process, a biphasic culture can be created and sustained by manipulating feeding strategies and media formulations to distinguish the period of cell proliferation to achieve a desired cell density (the growth phase) from the period of suspended or slow cell growth (the production phase). As such, fed-batch cultures have the potential to achieve higher product titers compared to batch cultures. Typically, a batch method is used during the growth phase and a fed-batch method is used during the production phase, but a fed-batch feeding strategy can be used throughout the entire process. However, unlike the batch process, bioreactor volume is a limiting factor which limits the amount of feed. Also, as with the batch method, metabolic by-product accumulation will lead to culture decline, which limits the duration of the production phase, often around 10 to 21 days. Fed-batch cultures are discontinuous, and harvest typically occurs when metabolic by-product levels or culture viability reach predetermined levels. When compared to a batch culture, in which no feeding occurs, a fed-batch culture can produce greater amounts of recombinant protein. (See, e.g., U.S. Pat. No. 5,672,502.)

Perfusion methods offer potential improvements over the batch and fed-batch methods by adding fresh media and simultaneously removing spent media during culture. A perfusion culture is one in which the cell culture receives fresh perfusion feed medium while simultaneously removing spent medium. Typical perfusion cultures begin with a batch culture start-up lasting for a day or two followed by continuous, step-wise, and/or intermittent addition of fresh feed media to the culture and simultaneous removal of spent media, with the retention of cells and additional high molecular weight compounds such as proteins (based on the filter molecular weight cutoff) throughout the growth and production phases of the culture. Various methods, such as sedimentation, centrifugation, or filtration, can be used to remove spent media while maintaining cell density. A non-limiting example of a filtration method is alternating tangential flow filtration. Alternating tangential flow is maintained by pumping medium through hollow-fiber filter modules. See e.g. U.S. Pat. No. 6,544,424; Furey, 2002, Gen. Eng. News. 22 (7): 62-63.

Perfusion can be continuous, stepwise, intermittent, or a combination of any or all of any of these. Perfusion rates can be less than a working volume to many working volumes per day. The cells are retained in the culture, and the spent medium that is removed is substantially free of cells or has significantly fewer cells than the culture. Recombinant proteins expressed by the cell culture can also be retained in the culture.

Typical large scale commercial cell culture strategies strive to reach high cell densities, 60-90(+)×10⁶ cells/mL, where almost a third to over one-half of the reactor volume is biomass. With perfusion culture, extreme cell densities of >1×10⁸ cells/mL have been achieved; even higher densities are predicted. A potential advantage of the perfusion process is that the production culture can be maintained for longer periods than batch or fed-batch culture methods. However, increased media preparation, use, storage, and disposal are necessary to support a long-term perfusion culture, particularly for a culture with high cell density, which also needs even more nutrients. All of this increases production costs compared to batch and fed-batch methods. In addition, higher cell densities can cause problems during production, such as, e.g., maintaining dissolved oxygen levels and problems with increased gassing, including supplying more oxygen and removing more carbon dioxide, which could result in more foaming and the need for alterations to antifoam strategies; as well as during harvest and downstream processing where the efforts required to remove the excessive cell material can result in loss of product, negating the benefit of increased titer due to increased cell mass.

An alternative large-scale cell culture strategy suitable for use in a flexible facility 10 of the present disclosure combines fed-batch feeding during the growth phase with continuous perfusion during the production phase. In some embodiments, a fed-batch culture with bolus feeds is used to maintain a cell culture during the growth phase. Perfusion feeding can then be used during a production phase. In some embodiments, perfusion begins when the cells have reached a production phase.

### Therapeutic Products

Various therapeutic products may be produced in a flexible facility according to the present disclosure. Polypeptides and proteins of interest, which represent a subset of therapeutic products, can be of scientific or commercial interest, including, e.g., protein-based therapeutics. Proteins of interest include, but are not limited to, secreted proteins, non-secreted proteins, intracellular proteins, or membrane-bound proteins. Polypeptides and proteins of interest can be produced by recombinant animal cell lines using cell culture methods and may be referred to as “recombinant proteins.” The expressed protein(s) may be produced intracellularly or secreted into the culture medium from which it can be recovered and/or collected. The term “isolated protein” or “isolated recombinant protein” refers to a polypeptide or protein of interest that is purified away from proteins or polypeptides or other contaminants that would interfere with its therapeutic, diagnostic, prophylactic, research, or other use.

Proteins of interest include, but are not limited to, proteins that exert a therapeutic effect by binding a target, such as, e.g., a target among those listed below, including targets derived therefrom, targets related thereto, and modifications thereof.

Proteins of interest may include, but are not limited to, “antigen-binding proteins.” An “antigen-binding protein” refers to a protein or polypeptide that comprises an antigen-binding region or antigen-binding portion that has affinity for another molecule to which it binds (antigen). Antigen-binding proteins include, but are not limited to, antibodies, peptibodies, antibody fragments, antibody derivatives, antibody analogs, fusion proteins (including, e.g., single-chain variable fragments (scFvs), double-chain (divalent) scFvs, and IgGscFv (see, e.g., Orcutt et al., 2010, Protein Eng Des Sel 23:221-228)), hetero-IgG (see, e.g., Liu et al., 2015, J Biol Chem 290:7535-7562), muteins, and XmAb® (Xencor, Inc., Monrovia, CA). Also included are bispecific T cell engagers (BiTE®), bispecific T cell engagers having extensions, such as half-life extensions, such as, e.g., HLE BiTEs, Heterolg BITE, and others, chimeric antigen receptors (CARs, CAR Ts), and T cell receptors (TCRs).

In some embodiments, proteins of interest may include colony stimulating factors, such as, e.g., granulocyte colony-stimulating factor (G-CSF). Such G-CSF agents include, but are not limited to, Neupogen® (filgrastim) and Neulasta® (pegfilgrastim). Also included are erythropoiesis stimulating agents (ESA), such as, e.g., Epogen® (epoetin alfa), Aranesp® (darbepoetin alfa), Dynepo® (epoetin delta), Mircera® (methyoxy polyethylene glycol-epoetin beta), Hematide®, MRK-2578, INS-22, Retacrit® (epoetin zeta), Neorecormon® (epoetin beta), Silapo® (epoetin zeta), Binocrit® (epoetin alfa), epoetin alfa Hexal, Abseamed® (epoetin alfa), Ratioepo® (epoetin theta), Eporatio® (epoetin theta), Biopoin® (epoetin theta), epoetin alfa, epoetin beta, epoetin zeta, epoetin theta, and epoetin delta, epoetin omega, epoetin iota, tissue plasminogen activator, and GLP-1 receptor agonists, as well as variants or analogs thereof and biosimilars of any of the foregoing.

In some embodiments, proteins of interest bind to one of more of the following, alone or in any combination: CD proteins including, but not limited to, CD3, CD4, CD5, CD7, CD8, CD19, CD20, CD22, CD25, CD30, CD33, CD34, CD38, CD40, CD70, CD123, CD133, CD138, CD171, and CD174, HER receptor family proteins, including, for instance, HER2, HER3, HER4, and the EGF receptor, EGFRvill, cell adhesion molecules, for example, LFA-1, Mol, p150,95, VLA-4, ICAM-1, VCAM, and alpha v/beta 3 integrin, growth factors, including but not limited to, for example, vascular endothelial growth factor (“VEGF”); VEGFR2, growth hormone, thyroid stimulating hormone, follicle stimulating hormone, luteinizing hormone, growth hormone releasing factor, parathyroid hormone, mullerian-inhibiting substance, human macrophage inflammatory protein (MIP-1-alpha), erythropoietin (EPO), nerve growth factor, such as NGF-beta, platelet-derived growth factor (PDGF), fibroblast growth factors, including, for instance, aFGF and bFGF, epidermal growth factor (EGF), Cripto, transforming growth factors (TGF), including, among others, TGF-α and TGF-β, including TGF-β1, TGF-β2, TGF-β3, TGF-β4, or TGF-β5, insulin-like growth factors-I and -II (IGF-I and IGF-II), des(1-3)-IGF-I (brain IGF-I), and osteoinductive factors, insulins and insulin-related proteins, including, but not limited to, insulin, insulin A-chain, insulin B-chain, proinsulin, and insulin-like growth factor binding proteins; (coagulation and coagulation-related proteins, such as, among others, factor VIII, tissue factor, von Willebrand factor, protein C, alpha-1-antitrypsin, plasminogen activators, such as urokinase and tissue plasminogen activator (“t-PA”), bombazine, thrombin, thrombopoietin, and thrombopoietin receptor, colony stimulating factors (CSFs), including the following, among others, M-CSF, GM-CSF, and G-CSF, other blood and serum proteins, including but not limited to albumin, IgE, and blood group antigens, receptors and receptor-associated proteins, including, for example, flk2/flt3 receptor, obesity (OB) receptor, growth hormone receptors, and T-cell receptors; neurotrophic factors, including but not limited to, bone-derived neurotrophic factor (BDNF) and neurotrophin-3,-4,-5, or -6 (NT-3, NT-4, NT-5, or NT-6); relaxin A-chain, relaxin B-chain, and prorelaxin, interferons, including for example, interferon-alpha,-beta, and -gamma, interleukins (ILs), e.g., IL-1 to IL-10, IL-12, IL-15, IL-17, IL-23, IL-12/IL-23, IL-2Ra, IL1-R1, IL-6 receptor, IL-4 receptor and/or IL-13 to the receptor, IL-13RA2, or IL-17 receptor, IL-1RAP; viral antigens, including but not limited to, an AIDS envelope viral antigen, lipoproteins, calcitonin, glucagon, atrial natriuretic factor, lung surfactant, tumor necrosis factor-alpha and -beta, enkephalinase, BCMA, IgKappa, ROR-1, ERBB2, mesothelin, RANTES (regulated on activation normally T-cell expressed and secreted), mouse gonadotropin-associated peptide, DNase, FR-alpha, inhibin, and activin, integrin, protein A or D, rheumatoid factors, immunotoxins, bone morphogenetic protein (BMP), superoxide dismutase, surface membrane proteins, decay accelerating factor (DAF), AIDS envelope, transport proteins, homing receptors, MIC (MIC-a, MIC-B), ULBP 1-6, EPCAM, addressins, regulatory proteins, immunoadhesins, antigen-binding proteins, somatropin, CTGF, CTLA4, eotaxin-1, MUC1, CEA, c-MET, Claudin-18, GPC-3, EPHA2, FPA, LMP1, MG7, NY-ESO-1, PSCA, ganglioside GD2, ganglioside GM2, BAFF, OPGL (RANKL), myostatin, Dickkopf-1 (DKK-1), Ang2, NGF, IGF-1 receptor, hepatocyte growth factor (HGF), TRAIL-R2, c-Kit, B7RP-1, PSMA, NKG2D-1, programmed cell death protein 1 and ligand, PD1 and PDL 1, mannose receptor/hCGβ, hepatitis-C virus, mesothelin dsFv[PE38] conjugate, Legionella pneumophila (lly), IFN gamma, interferon gamma induced protein 10 (IP10), IFNAR, TALL-1, thymic stromal lymphopoietin (TSLP), proprotein convertase subtilisin/Kexin Type 9 (PCSK9), stem cell factors, Flt-3, calcitonin gene-related peptide (CGRP), OX40L, α4β7, platelet specific (platelet glycoprotein IIb/IIIb (PAC-1), transforming growth factor beta (TFGβ), Zona pellucida sperm-binding protein 3 (ZP-3), TWEAK, platelet derived growth factor receptor alpha (PDGFRα), sclerostin, and biologically active fragments or variants of any of the foregoing.

In some embodiments, proteins of interest include abciximab, adalimumab, adecatumumab, aflibercept, alemtuzumab, alirocumab, anakinra, atacicept, basiliximab, belimumab, bevacizumab, biosozumab, blinatumomab, brentuximab vedotin, brodalumab, cantuzumab mertansine, canakinumab, cetuximab, certolizumab pegol, conatumumab, daclizumab, denosumab, eculizumab, edrecolomab, efalizumab, epratuzumab, etanercept, evolocumab, galiximab, ganitumab, gemtuzumab, golimumab, ibritumomab tiuxetan, infliximab, ipilimumab, lerdelimumab, lumiliximab, Ixdkizumab, mapatumumab, motesanib diphosphate, muromonab-CD3, natalizumab, nesiritide, nimotuzumab, nivolumab, ocrelizumab, ofatumumab, omalizumab, oprelvekin, palivizumab, panitumumab, pembrolizumab, pertuzumab, pexelizumab, ranibizumab, rilotumumab, rituximab, romiplostim, romosozumab, sargamostim, tocilizumab, tositumomab, trastuzumab, ustekinumab, vedolizumab, visilizumab, volociximab, zanolimumab, and zalutumumab, as well as biosimilars of any of the foregoing.

In some embodiments, proteins of interest can also include genetically engineered receptors, such as, e.g., chimeric antigen receptors (CARs or CAR-Ts) and T-cell receptors (TCRs), as well as other proteins comprising an antigen binding molecule that interacts with that targeted antigen. CARs can be engineered to bind to an antigen (such as, e.g., a cell-surface antigen) by incorporating an antigen-binding molecule that interacts with that targeted antigen. CARs typically incorporate an antigen binding domain (such as scFv) in tandem with one or more costimulatory (“signaling”) domains and one or more activating domains.

All documents, or portions of documents, cited in this application, including but not limited to patents, patent applications, articles, books, and treatises, are hereby expressly incorporated by reference. What is described in an embodiment of the disclosure can be combined with one or more other embodiments of the disclosure unless context clearly indicates otherwise.

The disclosed subject matter is not intended to be limited in scope by the specific embodiments described herein, which are instead intended as non-limiting illustrations of individual aspects of the disclosure. Functionally equivalent methods and components are within the scope of the disclosure. Indeed, various modifications of the disclosed subject matter, in addition to those shown and described herein, will be apparent to those skilled in the art from the foregoing description and accompanying drawings. Such modifications are intended to fall within the scope of the disclosed subject matter.

The descriptions of the various embodiments and/or examples of the disclosed subject matter have been presented for purposes of illustration, but are not intended to be exhaustive or limiting in any way. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, and/or to enable others of ordinary skill in the art to understand the disclosed subject matter.

1. A flexible facility for manufacturing at least one therapeutic product, comprising:
a reconfigurable, single-use area comprising at least one single-use bioreactor, wherein one or more of the at least one single use bioreactor(s) has a volume of at least 1000 L; and
a stainless steel area comprising at least one stainless steel bioreactor, wherein each stainless steel bioreactor in the stainless steel area has a volume greater than 5000 L, wherein one or more of the at least one stainless steel bioreactor(s) is fluidly connected to one or more of the at least one single use bioreactor(s).

2. The flexible facility according to claim 1, wherein one or more of the at least one single use bioreactor(s) has a volume of at least 2000 L.

3. The flexible facility according to either claim 1, wherein the stainless steel area comprises a plurality of stainless steel bioreactors, each having a volume of at least 10,000 L.

4. The flexible facility according to claim 1, wherein the reconfigurable, single-use area and the stainless steel area are segregated by a partition.

5. The flexible facility according to claim 4, wherein the partition is a wall.

6. The flexible facility according to claim 5, wherein the stainless steel area is completely enclosed with separate air handling.

7. The flexible facility according to claim 5, wherein a portion of the at least one stainless steel bioreactor(s) penetrates the wall and is fluidly connected through tubing to one or more of the at least one single use bioreactor(s).

8. The flexible facility according to claim 7, wherein the portion penetrating the wall comprises one or more components independently chosen from probe belts, connections for perfusion, and feed solution tank connections.

9. The flexible facility according to claim 1, wherein the fluid connection is configured to enable bypass of the stainless steel area.

10. The flexible facility according to claim 1, wherein the reconfigurable, single-use area and/or the stainless steel area further comprises one or more centrifuges.

11. The flexible facility according to claim 1, wherein the reconfigurable, single-use area comprises one or more seed trains.

12. The flexible facility according to claim 1, wherein the reconfigurable, single-use area further comprises one or more purification skids.

13. The flexible facility according to claim 12, wherein each of the one or more purification skids comprises one or more components independently chosen from chromatography skids, chromatography columns, viral inactivation systems, viral filtration systems, ultrafiltration diafiltration systems, and bulk filtration systems.

14. The flexible facility according to claim 1, further comprising one or more additional spaces independently chosen from utility spaces, media buffer facilities, personnel changing rooms, and production units.

15. The flexible facility according to claim 1, wherein the flexible facility is configured for producing one or more therapeutic products independently chosen from monoclonal antibodies, BiTE® molecules, RNA-based drug substances, cell therapies, and fermentation products.

16. The flexible facility according to claim 1, wherein the flexible facility is configured to implement one or more processes independently chosen from mammalian cell culture fed-batch processes, mammalian cell culture perfusion processes, continuous mammalian cell culture processes, microbial fermentation fed-batch processes, and combinations of any of the foregoing.
